SEQUENCE LISTING (6 sequences)

SEQ ID NO: 1 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGETKCQHQQVNESEPVYLEVFSDWLLLQASAEVVMEGQPLFLRCHGWRNWDVTKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 2 - protein, 232 aa, Homo sapiens
VPQKPKVSLNPPWNRIFKGENVTLTCNGNNFFEVSSTKWFHNGSLSEETNSSLNIVNAKFEDSGETKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 3 - protein, 180 aa, Homo sapiens
VPQKPKVSLNPPWNRIFKGENVTLTCNGNNFFEVSSTKWFHNGSLSEETNSSLNIVNAKFEDSGETKCQHQQVNESEPVYLEVFSDWLLLQASAEVVMEGQPLFLRCHGWRNWDVYKVIYYKDGEALKYWYEDHNISITNATVEDSGTYYCTGKVWQLDYESEPLNITVIKAPREKYWLQ

SEQ ID NO: 4 - protein, 199 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFI

SEQ ID NO: 5 - protein, 172 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKA

SEQ ID NO: 6 - protein, 197 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKA

(19) United States
(12) Patent Application Publication
(10) Pub. No.: US 2011/0256641 A1
(43) Pub. Date: Oct. 20, 2011

(54) Methods and Systems for Detecting Free IgE

(76) Inventors: Michael Ling, Toronto (CA); Mark Stephen Colwell, Olathe, KS (US); Michelle Lee Altrich, Lenexa, KS (US)

(21) Appl. No.: 13/078,746

(22) Filed: Apr. 1, 2011

Related U.S. Application Data
(60) Provisional application No. 61/325,711, filed on Apr. 19, 2010.

(51) Int. Cl. G01N 33/544

(52) U.S. Cl. 436/513

(57) ABSTRACT

The present disclosure provides methods, systems, and kits for detecting IgE antibodies available to bind to the Fc epsilon receptor in a biological sample from a subject receiving anti-IgE therapy. These methods, systems, and kits find use in monitoring anti-IgE therapy and determining its efficacy.

### CROSS REFERENCE

This application claims the benefit of U.S. Provisional Patent Application No. 61/325,711, filed Apr. 19, 2010, which application is incorporated herein by reference in its entirety.

### INTRODUCTION

The epsilon immunoglobulin (IgE) antibody frequently plays a role in diseases such as allergy, atopic disease, hyper IgE syndrome, asthma, and the like. IgE antibodies bind to a large variety of allergens. Each IgE molecule has a single allergen specificity. Once allergen bound, IgE antibodies can trigger immunological and allergic reactions, for example, via basophil activation. The treatments of such diseases can involve reducing the levels of IgE. Such treatments may accomplish this by, for example, reducing the production of IgE, for example, by allergen avoidance, pharmacotherapy, immunotherapy, and the like. Alternatively/in addition, such treatments may reduce the activity of IgE antibody by blocking its binding to Fc epsilon receptors.

Omalizumab (Xolair®), a recombinant humanized IgG1 monoclonal anti-human IgE Fc antibody is used to reduce the activity of IgE antibody by blocking it's binding to Fc epsilon receptors. Omalizumab is an anti-IgE antibody therapeutic to treat moderate to severe persistent allergic asthma.

Detection of IgE antibodies that are available to bind to Fc epsilon receptors can be used to monitor such therapy and to ascertain the efficacy of the therapy.

### SUMMARY

The present disclosure provides methods, systems, and kits for detecting free IgE antibodies in a biological sample from a subject receiving therapies that alter the ability of IgE to interact with Fc epsilon receptors (anti-IgE). These methods, systems, and kits find use in monitoring these therapies and determining their efficacy.

### BRIEF DESCRIPTION OF THE DRAWINGS

FIGS. 1A-1D depict absorbance measured with serial dilutions of the indicated samples in wells coated with the indicated concentrations of biotinylated FcεRIα.

FIGS. 2A-2C illustrate that IgE concentrations measured using the immobilized biotinylated FcεRIα polypeptide (observed IU/ml) correlated with the known IgE concentrations (expected IU/ml) for serial dilutions of the three samples (sample 1-3).

FIG. 3 shows the agreement between the IgE levels measured using immobilized biotinylated FcεRIα polypeptide (Free IgE) and immobilized anti-IgE antibody (Total IgE) in samples.

FIG. 4 illustrates that the levels of total IgE does not change with increasing concentrations of Omalizumab as measured by using immobilized anti-IgE antibody.

FIG. 5 illustrates that the levels of free IgE decreases with increasing concentration of Omalizumab as measured by using immobilized biotinylated FcεRIα polypeptide.

### DETAILED DESCRIPTION OF EXEMPLARY EMBODIMENTS

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an anchoring compound” includes a plurality of anchoring compounds, these anchoring compounds may be the same or different.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

It is appreciated that certain features of the methods, compositions, and systems disclosed herein, which are, for clarity, described in the context of separate embodiments, may also be provided in combination in a single embodiment. Conversely, various features of the methods, compositions, and systems disclosed herein, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The phrase “anti-IgE therapeutic” refers to an agent that is capable of binding to the Fc region, specifically the CH3 domain, of IgE antibody thereby preventing the IgE antibody from binding to FcεRI receptor present on mast cells and basophils. Examples of anti-IgE therapeutics include anti-IgE antibodies that bind to the Fc region, e.g., CH3 region of the IgE antibody, IgE binding fragments of FcεRI receptor, and the like. Anti-IgE therapeutic is also referred to as anti-IgE antibody therapeutic.

The phrases “free IgE antibody” or “free IgE” refers to an IgE antibody that is capable of binding to FcεRI receptor. Free IgE as used herein may or may not be bound to an antigen, e.g., an allergen. Free IgE refers to all free IgE regardless of allergen specificity.

The phrases “bound IgE antibody” or “IgE-anti-IgE therapeutic complex” or “IgE-anti-IgE complex” refers to an IgE antibody that is bound to an anti-IgE therapeutic such that the IgE is no longer able to bind to the FcεRI receptor.

The phrase “total IgE antibody” refers to the sum of free IgE antibodies and bound IgE antibodies present in a sample.

The terms “individual,” “subject,” “host,” and “patient,” are used interchangeably herein and refer to any human subject for whom monitoring of therapy is desired.

Overview

The methods of the present disclosure utilize at least an IgE Fc binding portion of a FcεRIα polypeptide immobilized on a substrate via an anchoring compound as a capture reagent to specifically bind free IgE antibodies, i.e., IgE antibodies that are not bound by an anti-IgE therapeutic. The methods of the present disclosure enable detection of free IgE antibodies in an undiluted biological sample obtained from a subject receiving anti-IgE therapy. The present disclosure provides assay methods that can avoid matrix effect. Matrix effect is a phenomenon where the level of the free IgE antibody detected in an undiluted sample does not directly correlate with that detected in diluted aliquots of the same sample.

### Method for Detecting Free IgE Antibody in Biological Sample

As noted above, a method for detecting free IgE level in a biological sample from a patient receiving an anti-IgE therapy is provided. The method includes: (a) contacting the biological sample with a capture reagent comprising at least an Fc binding portion of FcεRIα polypeptide under conditions suitable for binding of free IgE antibody to the capture reagent thereby generating a free IgE antibody-capture reagent complex, wherein the capture reagent is immobilized onto a substrate via an anchoring molecule that is directly or indirectly attached to the substrate; (b) contacting the capture reagent-free IgE antibody complex with a detection reagent that binds to a region of the free IgE not bound by the capture reagent; and (c) detecting the detection reagent bound to the capture reagent-free IgE antibody complex thereby detecting the free IgE antibody in the biological sample.

The assays of the present disclosure can be sufficiently sensitive to accurately measure very low levels of IgE antibody, especially the low levels of free IgE antibody that are present in a biological sample from a subject receiving anti-IgE therapy. In certain embodiments, this assay can measure free IgE present at levels of about 10 IU/ml or less, such as, 9 IU/ml, 8 IU/ml, 7 IU/ml, 6 IU/ml, 5 IU/ml, 4 IU/ml, 3 IU/ml, 2 IU/ml, 2.5 IU/ml, or 1 IU/ml. In certain embodiments, this assay can measure free IgE antibody levels in a sample in which the free IgE antibody is only about 1% of the total IgE antibody present in the sample. In related embodiments, this assay can measure free IgE antibody levels in a sample in which the free IgE antibody is only 0.1%, or 0.5%, or 1%, or 2%, or 3%, or 4%, or 5%, or more of the total IgE antibody present in the sample.

The method presented herein involves the use of a capture reagent that binds to free IgE antibodies but not to bound IgE antibodies (i.e., IgE antibodies that are bound to an anti-IgE therapeutic) present in a biological sample from a subject receiving anti-IgE therapy. The contacting of the biological sample to the capture reagent results in the formation of a free IgE antibody-capture reagent complex. This complex can be detected by a detection reagent. This detection reagent binds to a region of IgE antibody that is not bound by the capture reagent or by an antigen. The detecting is performed by reading a signal generated by the detection reagent. The signal may be read by any means for detecting a signal, such as a spectrophotomer, a fluorescent sample reader, etc. As explained in detail below, the detection reagent may produce a signal directly or indirectly. An example of a detection reagent that produces a signal directly is a detection reagent comprising a fluorescent moiety. An example of a detection reagent that produces a signal indirectly is a detection reagent comprising an enzyme, which converts its substrate into a detectable product. The capture reagent may be immobilized to a substrate by an anchoring molecule which directly or indirectly attaches the capture reagent to the substrate.

Components of the method of the present disclosure as well as formats for performing the methods are provided in the following sections.

Capture Reagents

Capture reagents comprising at least an IgE Fc binding portion of the FcεRIα polypeptide are utilized in the methods of the present disclosure.

IgE Fc binding portion (or Fc binding portion) of the FcεRIα polypeptide includes any region of the FcεRIα chain of FcεRI receptor that mediates binding to IgE antibody Fc region, specifically, CH3 (Cε3) domain of IgE antibody.

Fc binding portion of the FcεRIα polypeptide may include the amino acid sequence of human FcεRIα or an Fc binding portion thereof. Human FcεRIα is provided herewith (SEQ ID NO: 1). Human FcεRIα polypeptide (Accession No. NP 001992) has 257 amino acids. Amino acids 1-25 form the signal peptide. The mature protein is from residues 26-205, where amino acids 26-205 are predicted to be present in the extracellular region of the protein, amino acids 206-224 are predicted to form the trans-membrane region and amino acids 225-257 form the cytoplasmic domain. The extracellular region has two immunoglobulin G (IgG)-like domains.

In certain embodiments, the Fc binding portion of the FcεRIα polypeptide includes the mature (i.e., lacking the signal peptide) full-length human FcεRIα polypeptide (232 amino acids long; SEQ ID NO: 2). In certain embodiments, the Fc binding portion of FcεRIα polypeptide may be a human FcεRIα polypeptide lacking the signal peptide as well as the transmembrane and the cytoplasmic region (180 amino acids long; SEQ ID NO: 3). In certain embodiments, the Fc binding portion of FcεRIα polypeptide can be a human FcεRIα polypeptide lacking the signal peptide and the cytoplasmic region but having the transmembrane (199 amino acids long; SEQ ID NO: 4). In certain embodiments, the Fc binding portion of FcεRIα polypeptide can be a human FcεRIα polypeptide lacking the signal peptide as well as the transmembrane and the cytoplasmic region and having a shortened extracellular region (172 amino acids long; SEQ ID NO: 5). In certain embodiments, the Fc binding portion of FcεRIα polypeptide can be a human FcεRIα polypeptide with the signal peptide but lacking the transmembrane and the cytoplasmic region and having a shortened extracellular region (197 amino acids long; SEQ ID NO: 6).

In certain embodiments, the Fc binding portion may be obtained from a FcεRIα polypeptide, particularly human FcεRIα polypeptide, where the FcεRIα polypeptide comprises an amino acid sequence having at least about 80%, at least about 85%, at least about 90%, at least about 95%, at least about 98%, at least about 99%, or 100%, amino acid identity to a contiguous stretch of from about 100 amino acids (aa) to about 150 aa, from about 150 aa to about 200 aa, from about 200 aa to about 250 aa, from about 250 aa to about 250 aa, up to the full length, of an amino acid sequence provided in SEQ ID NO: 1. In certain embodiments, the Fc binding portion may be obtained from a FcεRIα polypeptide, particularly human FcεRIα polypeptide, where the FcεRIα polypeptide comprises an amino acid sequence having at least about 80%, at least about 85%, at least about 90%, at least about 95%, at least about 98%, at least about 99%, amino acid identity to an amino acid sequence provided in SEQ ID NO: 2, or SEQ ID NO: 3, or SEQ ID NO: 4, or SEQ ID NO: 5, or SEQ ID NO: 6.

In certain embodiments, the Fc binding portion of FcεRIα polypeptide may be obtained from a “functional derivative” of a naturally occurring human FcεRIα polypeptide. A “functional derivative” of a native sequence polypeptide is a polypeptide having a qualitative biological property in common with a native sequence polypeptide, for example, the ability to specifically bind Fc region, specifically, CH3 domain of IgE (i.e., Cε3). The term “derivative” encompasses both amino acid sequence variants of polypeptide, covalent modifications, and fusions thereof.

Mutants of FcεRIα polypeptide may be generated by performing conservative substitutions which have substantially no effect on binding to IgE. By conservative substitutions is intended combinations such as those from the following groups: gly, ala; val, ile, leu; asp, glu; asn, gln; ser, thr; lys, arg; and phe, tyr. Amino acids that are not present in the same group are “substantially different” amino acids. In certain cases, the conserved residues may not be substituted and the substitutions limited to the non-conserved residues.

In certain embodiments, the capture reagent may be purified from an organism genetically modified to express the capture reagent. The capture reagent may be purified by well-known methods including ammonium sulfate or ethanol precipitation, acid extraction, anion or cation exchange chromatography, phosphocellulose chromatography, hydrophobic interaction chromatography, high performance liquid chromatography, affinity chromatography, protein G affinity chromatography, for example, hydroxyapatite chromatography and lectin chromatography, etc. In other embodiments, the capture reagent may be synthesized using well known polypeptide synthesis methods.

In certain embodiments, the capture reagent may be modified by covalent conjugation to an anchoring compound. Examples of such anchoring compounds are provided below.

Anchoring Compounds

An anchoring compound may be any non-genetically encoded compound that enables the attachment of the capture reagent as described above to a substrate. By non-genetically encoded is meant that the anchoring compound is not encoded by a gene sequence and is not derived from a gene sequence present in nature. In other words, the anchoring compound is not a protein or a polypeptide. Suitable anchoring compounds include chemical linkers, such as bi-functional linkers, a non-genetically encoded member of a binding pair, such as, biotin from the binding pair of biotin and avidin or streptavidin, and the like.

As noted above, the capture reagent may comprise an anchoring compound. The anchoring compound may be attached to the capture reagent post-translationally.

The anchoring compound may be attached to a substrate.

The anchoring compound may directly or indirectly immobilize the capture reagent to the substrate.

Examples of anchoring compounds that directly attach the capture reagent to a substrate include a bi-functional chemical linker molecule that can bind to a substrate and to the capture reagent. The anchoring compound may be first linked to the substrate and then to the capture reagent, or the anchoring compound may be first attached to the capture reagent and the capture reagent comprising the anchoring compound may be linked to the substrate, or the anchoring compound may be linked to the substrate and capture reagent simultaneously. Anchoring compounds as well as methods for using them are well known in the art. A bifunctional linker compound that may be used to attach capture reagent to a substrate is described in U.S. Pat. No. 5,063,109, for example. U.S. Pat. No. 5,063,109 provides anchoring compounds of solid phases with linking groups that can be used to link substrate to substances such as proteins. These anchoring compounds are hydrophilic so that they are quite stable in aqueous solutions and preserve the conformation of the proteins they link to the substrate. The anchoring compound may have formula:

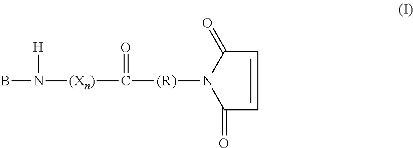

wherein B is an amine bearing solid phase material; X is a substituted or unsubstituted amino acid having from three to ten carbon atoms in a straight chain; n is from one to ten; and R is an alkyl, cycloalkyl, an alkyl cyclo-alkyl or an aromatic carbocyclic ring. The anchoring compound may have formula:

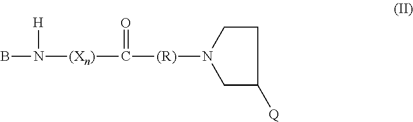

wherein Q is a SH or a thiol bearing peptide, polypeptide or protein; and wherein B, X, n, and R are as defined above. U.S. Pat. No. 5,063,109 is herein incorporated by reference for its description of conjugation molecules, methods of making and using the same.

Examples of anchoring compounds that indirectly link capture reagent to a substrate include anchoring molecules that are non-genetically encoded members of a binding pair. The anchoring molecule may be a first non-genetically encoded member of the binding pair and the second member of the binding pair may be attached to the substrate. The second member of the binding pair may be attached covalently or non-covalently to the substrate. Methods for attaching a protein to a substrate are well known in the art (see for example, The Immunoassay Handbook, David Wilde, 2005; Enzyme Immunoassays: From Concept to Product Development, S. S. Deshpande, 1996). For example, a binding partner such as avidin, streptavidin, or NeutrAvidin may be attached to a substrate via conventional means, and the capture reagent may be attached to biotin molecules via conventional means. The anchoring molecule biotin mediates the immobilization of capture reagent on the substrate via binding to its binding partner (e.g., avidin, streptavidin, or NeutrAvidin) attached to the substrate.

Biotin may be conjugated to capture reagent by a number of well known methods. Biotin is typically conjugated to proteins through primary amines (i.e., lysines) or carbohydrate groups. Biotin may be obtained from many commercial sources (such as Pierce EZ link Sulfo-NHS-LC biotin or Pierce NHS-LC biotin 11) and conjugated to capture reagent according to the manufacturer's instructions. Additionally, kits for biotin conjugation may be obtained from companies such as Sigma Aldrich, Alpha Diagnostic International, or Amersham Pharmacia Biotech. The amount of biotin conjugated to a polypeptide may be measured using a HABA kit. This testing of the biotinylated protein can be used to monitor consistency in biotin labeling of proteins between different batches of the biotinylated proteins.

Detection Reagent

The subject method involves detecting the binding of free IgE antibody to the capture reagent immobilized on a substrate via an anchoring molecule. This formation of free IgE antibody-capture reagent complex may be detected by a detection reagent that binds to the IgE antibody. This detection reagent binds to a region of IgE antibody not bound by the capture reagent and not bound by an antigen that binds to the IgE antibody. In other words, the detection reagent does not bind to the CH3 domain in the Fc region of IgE antibody and the epitope binding region of the IgE antibody. Use of a detection reagent that does not bind to the Cε3 domain in the Fc region of IgE antibody ensures that the detection reagent does not significantly compete with capture reagent for binding to the IgE antibody. Use of a detection reagent that does not bind to the antigen binding region of IgE antibody ensures that the binding moiety does not significantly compete with antigen bound to the IgE antibody for binding to the IgE antibody.

Suitable detection reagents include anti-IgE antibodies, such as anti-IgE monoclonal antibodies produced from clones HP6061 (mouse IgM anti-human IgE Fc) and HP6029 (mouse IgG1 anti-human IgE Fc). These antibodies can be obtained from EMD Biosciences Corporation (La Jolla, Calif.). Alternatively, polyclonal antiIgE antibodies can be used, such as from goat, rabbit etc. The detection antibodies are readily available from commercial sources, such as from Santa Cruz Biotechnology, Santa Cruz, Calif.).

The detection reagent may be labeled to facilitate detection free IgE antibody-capture reagent complex. Examples of labels include radiolabels, such as ³H or ¹²⁵I, fluorescent moieties, dyes, beads, chemiluminescent moieties, electrochemiluminescent moieties, colloidal particles, and the like. Useful labels include fluorochromes, e.g. Cy2, Cy3, Cy5, fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)). The detection reagent may be labeled with enzymes where the substrate may provide for a colored or fluorescent product. In a certain embodiment, the detection reagent is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Alternatively, the detection reagent may be unlabeled, and a labeled antibody that binds to detection reagent may be used. Since the resultant signal is thus amplified, this technique may be advantageous where only a small amount of IgE antibody is present.

The signal obtained from a label bound to the detection reagent may be read with suitable means such as a spectrophotometer, a fluorescent reader, ELISA plate reader etc. The amount of signal generated is proportional to the amount of free IgE antibody bound to the substrate (via the capture reagent).

Substrate

The methods of the present disclosure involve contacting a biological sample containing putative free IgE antibodies with a capture reagent immobilized to a substrate via an anchoring molecule.

Suitable substrates can have a variety of forms and compositions and can be derived from naturally occurring materials, naturally occurring materials that have been synthetically modified, or synthetic materials. Examples of suitable substrates include, but are not limited to, nitrocellulose, cellulose, glasses, silicas, teflons, and metals (for example, gold, platinum, and the like). Suitable materials for substrates that find use in the present methods also include polymeric materials, including plastics (for example, polytetrafluoroethylene, polypropylene, polystyrene, polycarbonate, and blends thereof, and the like), polysaccharides such as agarose and dextran, polyacrylamides, polystyrenes, polyvinyl alcohols, copolymers of hydroxyethyl methacrylate and methyl methacrylate, activated carbon and the like.

The substrate may be homogenous or a composite structure of two or more different materials, e.g., where the substrate includes a first base material that is coated on a surface with one or more additional different coating materials. Another example is the polystyrene beads used in immunoassays. Yet another example is the activated carbon surface which can accept proteins, peptides or other antigens or antibodies.

The substrate may have any desired configuration. The substrate can be, for example, solid or semisolid. In certain embodiments, the substrate is in the form of a substrate and/or wafer that includes a flat, planar surface. As such, the substrate may be a uniform substrate, e.g., a wafer of solid material, such as silicon, glass, quartz, polymerics, such as polycarbonate, polystyrene, etc., or can comprise additional elements, e.g., structural, compositional, etc. The substrate can be in the form of a bead (spherical) or a porous material, such as in a shape and texture of sponge to create a large assay surface area.

A substrate may be in the form of a plate with wells or microwells, porous or non-porous beads, beads packed into a column, beads in a well or microwell, tubes, cellulose matrix in a tube, etc.

Biological Sample

The biological sample may be any sample from a patient undergoing anti-IgE therapy. For example, a biological sample may include blood, plasma, serum, fractions of plasma, cerebrospinal fluid, synovial fluid, lymph, bronchial aspirates, and the like.

In many embodiments, a suitable initial source for the biological sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood allowed to clot and the serum separated and collected for the assay.

In embodiments in which the sample is a serum or serum derived sample, the sample is generally a fluid sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. In some cases, blood may be collected from a subject by venipuncture. 0.1-0.5 ml may be used to prepare serum or plasma. Serum may be prepared just after blood drawing. Tubes may be left at room temperature for 4 hours following centrifugation after which serum is removed. Serum may be aliquoted and stored at −20° C. Plasma may be prepared by adding EDTA (final concentration of 5 mM) to a blood sample. Blood sample may be centrifuged, supernatant removed and stored at −20° C.

In certain embodiments, the biological sample is not diluted, for example, the sample is not mixed with an aqueous phase. For the accurate determination of free IgE in a biological sample, it is important to maintain the equilibrium of free IgE, bound IgE and the anti-IgE therapeutic as it exists in the patient and the undiluted biological sample obtained from the patient. Once the biological sample is diluted, the equilibrium may be altered leading to an inaccurate determination of free IgE antibody levels in the patient.

The biological sample for the assay may be obtained from a patient receiving anti-IgE therapy at any time point before or after the start of the therapy. For example, the sample(s) may be obtained any time before, at day 1, day 2, day 3, day 4, day 5 day 6, week 1, week 2 week 3 week 4, week 5, week 6, week 7, week 8, week 9, week 10, week 11, week 12, week 13, week 14, week 15, week 16, week 17, week 18, week 19, week 20, or more after the start of the anti-IgE therapy. In some embodiments, the samples may be obtained at more than one of the foregoing time points.

Anti-IgE therapy includes treatment regimen in which a subject in need of an anti-IgE therapy is administered an anti-IgE therapeutic. The anti-IgE therapeutic may be any molecule that can bind to the Fc region of the IgE antibody thereby preventing the IgE antibody from binding to the FcεRI receptor. For example, an anti-IgE therapeutic may be anti-IgE antibody that bind's to the CH3 domain in the Fc region of IgE antibody or a FcεRI polypeptide or a fragment thereof that binds to the CH3 domain in the Fc region of IgE antibody.

Examples of an antibody that binds to the Fc region of Ig antibody preventing it from binding to the FcRI receptor on mast cells and basophils is Omalizumab (Xolair®). Omalizumab is a recombinant humanized IgG1 monoclonal antibody licensed for use in the United States to treat moderate to severe persistent allergic asthma.

Examples of a polypeptide that binds to the Fc region of Ig antibody preventing it from binding to the FcRI receptor on mast cells and basophils is a full-length FcεRI polypeptide or fragment thereof, where the fragment is capable of binding to the Fc region of Ig antibody preventing it from binding to the FcRI receptor on mast cells and basophils.

Assay Formats

As will be readily apparent, design of the assays described herein is subject to a great deal of variation, and many formats are known in the art. The following descriptions are merely provided as guidance and one of skill in the art can readily modify the described protocols, using techniques well known in the art.

Generally, the assay involves (a) combining a biological sample suspected of containing free IgE antibodies and a capture reagent comprising at least an Fc binding portion of FcεRIα polypeptide in a first reaction mixture under conditions suitable for binding of free IgE antibody to the capture reagent thereby generating a free IgE antibody-capture reagent complex, wherein the capture reagent is immobilized onto a substrate via an anchoring compound that is directly or indirectly attached to the substrate; (b) contacting the free IgE antibody-capture reagent complex obtained from the reaction with a detection reagent that binds to a region of the free IgE not bound by the capture reagent to produce a second reaction mixture; and (c) detecting the detection reagent bound to the capture reagent-free IgE antibody complex in the second reaction mixture thereby detecting the free IgE antibody in the sample.

Generally, the biological sample may be contacted with the capture reagent following immobilization of the capture reagent on the substrate. Before contacting with the biological sample, the substrate on which capture reagent is immobilized is incubated with a blocking solution to block any non-specific binding sites on the substrate, i.e., those sites not occupied by capture reagent. The blocking solution may include a buffer providing a physiological pH (e.g., PBS) and a blocking reagent such as bovine serum albumin, casein, gelatin, and the like. In some cases, the blocking reagent may also include a detergent at non-interfering concentrations, such as Tween, NP40, TX100, and the like.

The neat (i.e., undiluted) biological solution may be incubated with the capture reagent. For example, the undiluted serum sample from a subject receiving anti-IgE therapy may be contacted with the immobilized capture reagent.

After contacting the immobilized capture reagent with biological sample suspected of containing free IgE antibodies, free IgE-capture reagent complex may be generated. The presence of free IgE-capture reagent complex can be detected by a detection reagent.

Any IgE antibodies that are non-specifically bound to the substrate are washed away using a buffer containing a detergent at a concentration high enough disrupt any non-specific weak interactions but not to disrupt any specific interactions, for example, the binding of IgE to FcεRIα polypeptide.

After the washing step, the substrate may optionally be exposed to a blocking solution prior to adding a solution containing a detection reagent that specifically binds to IgE antibody. Any detection reagent that specifically binds to IgE antibody may be used as long as the detection reagent does not bind to the same region of IgE as bound by, capture reagent or an antigen, e.g., an allergen bound to IgE antibody. Various detection reagents that may be used in the assay are provided above.

After the detection reagent has bound, the insoluble support is generally again washed free of non-specifically bound molecules, and the binding of the detection reagent is detected, by for example, a signal produced by the detection reagent is detected by conventional means. Where a detection reagent conjugated to an enzyme is used, an appropriate enzyme substrate is provided so a detectable product is formed.

In certain embodiments, a report describing the findings of the subject assay may be generated and the subject method may include the step of generating a report summarizing the results of the contacting and detecting steps, such as, the presence or absence of free IgE antibody in the sample, levels of free IgE antibody in the sample, etc.

The signal obtained from a label bound to the detection reagent may be read with suitable means such as a spectrophotometer, a fluorescent reader, ELISA plate reader etc. The amount of signal generated is proportional to the amount of free IgE antibody bound to the substrate (via the capture reagent).

The signal detected can be compared to a control sample that provides a reference signal.

The reference signal may be a baseline signal. The baseline signal may be the signal obtained from a negative control, for example, a control containing no biological sample or an IgE antibody free sample (i.e., a sample known not to have any detectable levels of IgE), and the like.

A signal from the detection reagent that is similar to a baseline signal would indicate that there are no significant levels of free IgE antibodies present in the biological sample, i.e., a signal close to the baseline signal would indicate the absence of free IgE antibodies in the sample. A signal from the detection reagent that is significantly higher than a baseline level would indicate the presence of free IgE antibodies in the biological sample.

In other embodiments, a positive control may be used to generate a reference signal value and a signal (from the detection reagent bound to the free IgE-capture reagent complex) that is close to or above the reference signal indicates the presence of free IgE antibodies in the biological sample. The positive control may be a sample containing known amounts of IgE antibody.

The reference signal may be obtained from an assay conducted in parallel to the foregoing assay or may be previously determined.

The detection of free IgE antibodies in a biological sample from a subject receiving anti-IgE therapy may be qualitative or quantitative measurement.

A qualitative measurement may simply be the detection of presence or absence of free IgE antibodies.

In another embodiment, a qualitative measurement may involve comparing the signal obtained from the biological sample assayed by the disclosed method to the signal obtained from a biological sample from the same subject prior to the start of anti-IgE therapy. A decrease in the signal may indicate that the therapy is having an effect.

Measurement of Free IgE Antibody Levels

Quantification may be performed by a comparison of the signal obtained from the detection reagent bound to the free IgE-capture reagent complex to a standard curve. A standard curve may be created in parallel assays or may be previously created. A standard curve may be created by measuring the signal obtained from known amounts of IgE. In an exemplary embodiment, at least about 5, 6, 7, or 8 concentrations of IgE may be measured to generate a standard curve. The concentration of free IgE in the sample may be measured using linear regression between concentrations versus absorbance of the standards.

Monitoring Anti-IgE Therapy Efficacy

The method, system, and kit of the present disclosure may be used to assess efficacy of an anti-IgE therapeutic. Efficacy of an anti-IgE therapeutic may be determined by a percent of free IgE, calculated by the following formula.

% of free IgE=free IgE/Total IgE

Total IgE may be measured by a number of methods known in the art. For example, ImmunoCAP 250 (Pharmacia, Kalamazoo, Mich.) is an FDA-approved solid phase immunometric assay in which an anti-IgE antibody is attached to a CAP matrix. This antibody binds to all the IgE antibodies present in a biological sample (i.e., free IgE and IgE bound by an anti-IgE therapeutic). The bound IgE may then be detected with an anti-IgE antibody, such as with detectably labeled anti-human IgE antibody.

A low % of free IgE, such as less than or equal to 20%, or less than 10%, or less than 5%, or lesser, indicates that the anti-IgE therapy is effective.

Alternatively, the total IgE level measured with a standard assay may be divided by the free IgE level measured using the method of the present disclosure. A ratio of 1 (i.e., 1:1) or close to 1 indicates that the anti-IgE therapy is not effective and that an increase in the dosage (for example by increasing the amount of anti-IgE therapeutic administered and/or number of times the therapeutic is administered) is warranted.

The efficacy of anti-IgE therapy may also be assessed by evaluating the concentration of free IgE. In general, for subjects with pre-dosing total IgE of 30-700 IU/ml or the like, a free IgE concentration of less than 10 IU/ml, represents a good outcome with respect to the reduction of free IgE by an anti-IgE therapeutic.

Reports

The methods of the present disclosure can include generating a report of the result of the method of detecting free IgE in a biological sample from a patient undergoing anti-IgE therapy. Such report may include whether free IgE antibodies were detected and/or concentration of the free IgE antibodies.

In certain embodiments, the report may also include the levels of total IgE present in the same sample.

In certain embodiments, the report may also include percent of free IgE as provided above or indicate the percent of free IgE present in the sample with respect to the total IgE.

A “report,” as described herein, is an electronic or tangible document. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) percent of free IgE, and 6) other features.

The methods disclosed herein can further include a step of generating or outputting a report as described above, which report can be provided in the form of an electronic medium (e.g., an electronic display on a computer monitor), or in the form of a tangible medium (e.g., a report printed on paper or other tangible medium).

For clarity, it should be noted that the term “user,” which is used interchangeably with “client,” is meant to refer to a person or entity to whom a report is transmitted, and may be the same person or entity who does one or more of the following: a) collects a sample; b) processes a sample; c) provides a sample or a processed sample; and d) generates data. In some cases, the person(s) or entity(ies) who provides sample collection and/or sample processing and/or data generation, and the person who receives the results and/or report may be different persons, but are both referred to as “users” or “clients” herein to avoid confusion.

In certain embodiments, e.g., where the methods are executed on a single computer, the user or client provides for data input and review of data output. A “user” can be a health professional (e.g., a clinician, a laboratory technician, a physician (e.g., a primary care physician), etc.).

In embodiments where the user only executes a portion of the method, the individual who reviews data output (e.g., results prior to release to provide a complete report, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance, sample collection ethical reviews and consent forms), all results, whether generated wholly or partially electronically, are subjected to a quality control and regulatory routine prior to release to the user.

Computer-Based Systems and Methods

The methods described herein can be implemented in numerous ways. In one embodiment of particular interest, the methods involve use of a communications infrastructure, for example, the interne. It is also to be understood that the present methods may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site associated (e.g., at a service provider's facility). In some embodiments, the step of calculating the levels of free IgE antibody or calculating the percent of free IgE is performed by a computer programmed to execute an algorithm for the calculation (e.g., calculating concentration by comparison to a standard curve). In other examples, the subject method includes causing a computer to execute an algorithm for calculating.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which can include test data; and may also include other data such as patient data. This information received can be stored at least temporarily in a database, and data analyzed to generate a report as described above.

Part or all of the input and output data can also be sent electronically; certain output data (e.g., reports as described above) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record which may exist in a confidential database at the healthcare facility.

Computer-Readable Storage Media

The present disclosure also contemplates a computer-readable storage medium (e.g. CD-ROM, memory key, flash memory card, diskette, etc.) having stored thereon a program which, when executed in a computing environment, provides for implementation of algorithms to carry out all or a portion of, for example, the calculation of levels of free IgE antibodies and/or percent of free IgE, as described herein. Where the computer-readable medium contains a complete program for carrying out the calculations required to determine the levels (e.g., concentration of free IgE and optionally total IgE), the program includes program instructions for collecting, analyzing and generating output, and generally includes computer readable code devices for interacting with a user as described herein, processing that data in conjunction with analytical information, and generating unique printed or electronic media for that user.

Where the storage medium provides a program which provides for implementation of a portion of the methods described herein (e.g., the user-side aspect of the methods (e.g., data input, report receipt capabilities, etc.)), the program provides for transmission of data input by the user (e.g., via the interne, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out the assaying step of the subject method (e.g., polypeptides, antibodies, or other such kit components).

### Systems

Also provided herein are systems for detecting free IgE antibody. The assay system may comprise a capture reagent comprising at least an IgE Fc binding portion of FcεRIα polypeptide immobilized on a substrate via an anchoring compound and a biological sample from a subject receiving anti-IgE therapy.

In certain embodiments, the system may further comprise a detection reagent that binds to a region of the free IgE antibody not bound by the capture reagent and an antigen.

The individual components of the system are as described above.

These systems and the methods for detecting the presence or absence of free IgE antibody as well as for measuring levels of free IgE antibody may be used for monitoring anti-IgE therapy and determining efficacy of anti-IgE therapy. The results from the methods disclosed herein may be used in determining modulation to therapy regimen.

### Kits

The materials for use in the methods of the present disclosure are suited for preparation of kits produced in accordance with well known procedures. The present disclosure thus provides kits comprising one or more capture reagents disclosed above as well as one or more detection reagents that specifically bind to IgE antibody that is bound to capture reagent and may also be bound to an antigen. In addition, the kits may optionally comprise the reagent(s) with an identifying description or label or instructions relating to their use in the methods of the present disclosure. The kits may comprise containers, each with one or more of the various reagents (typically in concentrated form) utilized in the methods, including, for example, a capture reagent comprising an anchoring compound. The kits may also comprise the multiwell plates, in which the capture reagent is immobilized via an anchoring compound, for contacting samples with assay reagents.

Mathematical algorithms used to estimate or quantify presence of free IgE antibodies, percent of free IgE, etc., are also properly potential components of kits.

### Sequences

The following are sequences referred to herein by SEQ ID NO.

```
SEQ ID NO: 1
Met Ala Pro Ala Met Glu Ser Pro Thr Leu Leu Cys Val Ala Leu Leu
  1 5 10 15
Phe Phe Ala Pro Asp Gly Val Leu Ala Val Pro Gln Lys Pro Lys Val
                        20 25 30
Ser Leu Asn Pro Pro Trp Asn Arg Ile Phe Lys Gly Glu Asn Val Thr
                35 40 45
Leu Thr Cys Anh Gly Asn Asn Phe Phe Glu Val Ser Ser Thr Lys Trp
        50 55 60
Phe His Asn Gly Ser Leu Ser Glu Glu Thr Asn Ser Ser Leu Asn Ile
65 70 75 80
Val Asn Ala Lys Phe Glu Asp Ser Gly Glu Tyr Lys Cys Glu His Ala
                                85 90 95
Gln Val Asn Glu Ser Glu Pro Val Tyr Leu Glu Val Phe Ser Asp Trp
                        100 105 110
Leu Leu Leu Gln Ala Ser Ala Glu Val Val Met Glu Gly Glu Pro Leu
                115 120 125
Phe Leu Arg Cys His Gly Trp Arg Asn Trp Asp Val Tyr Lys Val Ile
        130 135 140
Tyr Tyr Lys Asp Gly Glu Ala Leu Lys Tyr Trp Tyr Glu Asn His Asn
145 150 155 160
Ile Ser Ile Thr Asn Ala Thr Val Glu Asp Ser Gly Thr Tyr Tyr Cys
                                165 170 175
Thr Gly Lys Val Trp Gln Leu Asp Tyr Glu Ser Glu Pro Leu Asn Ile
                        180 185 190
Thr Val Ile Lys Ala Pro Arg Glu Lys Tyr Trp Leu Gln Phe Phe Ile
                195 200 205
Pro Leu Leu Val Val Ile Leu Phe Ala Val Asp Thr Gly Leu Phe Ile
        210 215 220
Ser Thr Gln Gln Gln Val Thr Phe Leu Leu Lys Ile Lys Arg Thr Arg
225 230 235 240
Lys Gly Phe Arg Leu Leu Asn Pro His Pro Lys Pro Asn Pro Lys Asn
                                245 250 255
Asn
SEQ ID NO: 2
Val Pro Gln Lys Pro Lys Val Ser Leu Asn Pro Pro Trp Asn Arg Ile
  1 5 10 15
Phe Lys Gly Glu Asn Val Thr Leu Thr Cys Asn Gly Asn Asn Phe Phe
                        20 25 30
Glu Val Ser Ser Thr Lys Trp Phe His Asn Gly Ser Leu Ser Glu Glu
                35 40 45
Thr Asn Ser Ser Leu Asn Ile Val Asn Ala Lys Phe Glu Asp Ser Gly
        50 55 60
Glu Tyr Lys Cys Gln His Gln Gln Val Asn Glu Ser Glu Pro Val Tyr
65 70 75 80
Leu Glu Val Phe Ser Asp Trp Leu Leu Leu Gln Ala Ser Ala Glu Val
                                85 90 95
Val Met Glu Gly Gln Pro Leu Phe Leu Arg Cys His Gly Trp Arg Asn
                        100 105 110
Trp Asp Val Tyr Lys Val Ile Tyr Tyr Lys Asp Gly Glu Ala Leu Lys
                115 120 125
Tyr Trp Tyr Glu Asn His Asn Ile Ser Ile Thr Asn Ala Thr Val Glu
        130 135 140
Asp Ser Gly Thr Tyr Tyr Cys Thr Gly Lys Val Trp Gln Leu Asp Tyr
145 150 155 160
Glu Ser Glu Pro Leu Asn Ile Thr Val Ile Lys Ala Pro Arg Glu Lys
                                165 170 175
Tyr Trp Leu Gln Phe Phe Ile Pro Leu Leu Val Val Ile Leu Phe Ala
                        180 185 190
Val Asp Thr Gly Leu Phe Ile Ser Thr Gln Gln Gln Val Thr Phe Leu
                195 200 205
Leu Lys Ile Lys Arg Thr Arg Lys Gly Phe Arg Leu Leu Asn Pro His
        210 215 220
Pro Lys Pro Asn Pro Lys Asn Asn
225 230
SEQ ID NO: 3
Val Pro Gln Lys Pro Lys Val Ser Leu Asn Pro Pro Trp Asn Arg Ile
1 5 10 15
Phe Lys Gly Glu Asn Val Thr Leu Thr Cys Asn Gly Asn Asn Phe Phe
                        20 25 30
Glu Val Ser Ser Thr Lys Trp Phe His Asn Gly Ser Leu Ser Glu Glu
                35 40 45
Thr Asn Ser Ser Leu Asn Ile Val Asn Ala Lys Phe Glu Asp Ser Gly
        50 55 60
Glu Tyr Lys Cys Gln His Gln Gln Val Asn Glu Ser Glu Pro Val Tyr
65 70 75 80
Leu Glu Val Phe Ser Asp Trp Leu Leu Leu Gln Ala Ser Ala Glu Val
                                85 90 95
Val Met Glu Gly Gln Pro Leu Phe Leu Arg Cys His Gly Trp Arg Asn
                        100 105 110
Trp Asp Val Tyr Lys Val Ile Tyr Tyr Lys Asp Gly Glu Ala Leu Lys
                115 120 125
Tyr Trp Tyr Glu Asn His Asn Ile Ser Ile Thr Asn Ala Thr Val Glu
        130 135 140
Asp Ser Gly Thr Tyr Tyr Cys Thr Gly Lys Val Try Gln Leu Asp Tyr
145 150 155 160
Glu Ser Glu Pro Leu Asn Ile Thr Val Ile Lys Ala Pro Arg Glu Lys
                                165 170 175
Tyr Trp Leu Gln
                        180
SEQ ID NO: 4
Val Pro Gln Lys Pro Lys Val Ser Leu Asn Pro Pro Trp Asn Arg Ile
  1 5 10 15
Phe Lys Gly Glu Asn Val Thr Leu Thr Cys Asn Gly Asn Asn Phe Phe
                        20 25 30
Glu Val Ser Ser Thr Lys Trp Phe His Asn Gly Ser Leu Ser Glu Glu
                35 40 45
Thr Asn Ser Ser Leu Asn Ile Val Asn Ala Lys Phe Glu Asp Ser Gly
        50 55 60
Glu Tyr Lys Cys Gln His Gln Gln Val Asn Glu Ser Glu Pro Val Tyr
65 70 75 80
Leu Glu Val Phe Ser Asp Trp Leu Leu Leu Gln Ala Ser Ala Glu Val
                                85 90 95
Val Met Glu Gly Gln Pro Leu Phe Leu Arg Cys His Gly Trp Arg Asn
                        100 105 110
Trp Asp Val Tyr Lys Val Ile Tyr Tyr Lys Asp Gly Glu Ala Leu Lys
                115 120 125
Tyr Trp Tyr Glu Asn His Asn Ile Ser Ile Thr Asn Ala Thr Val Glu
        130 135 140
Asp Ser Gly Thr Tyr Tyr Cys Thr Gly Lys Val Trp Gln Leu Asp Tyr
145 150 155 160
Glu Ser Gln Pro Leu Asn Ile Thr Val Ile Lys Ala Pro Arg Glu Lys
                                165 170 175
Tyr Trp Leu Gln Phe Phe Ile Pro Leu Leu Val Val Ile Leu Phe Ala
                        180 185 190
Val Asp Thr Gly Leu Phe Ile
                195
SEQ ID NO: 5
Val Pro Gln Lys Pro Lys Val Ser Leu Asn Pro Pro Trp Asn Arg Ile
  1 5 10 15
Phe Lys Gly Glu Asn Val Thr Leu Thr Cys Asn Gly Asn Asn Phe Phe
                        20 25 30
Glu Val Ser Ser Thr Lys Trp Phe His Asn Gly Ser Leu Ser Glu Glu
                35 40 45
Thr Asn Ser Ser Leu Asn Ile Val Asn Ala Lys Phe Glu Asp Ser Gly
        50 55 60
Glu Tyr Lys Cys Gln His Gln Gln Val Asn Glu Ser Glu Pro Val Tyr
65 70 75 80
Leu Glu Val Phe Ser Asp Trp Leu Leu Leu Gln Ala Ser Ala Glu Val
                                85 90 95
Val Met Gln Gly Gln Pro Leu Phe Leu Arg Cys His Gly Trp Arg Asn
                        100 105 110
Trp Asp Val Tyr Lys Val Ile Tyr Tyr Lys Asp Gly Glu Ala Leu Lys
                115 120 125
Tyr Trp Tyr Glu Asn His Asn Ile Ser Ile Thr Asn Ala Thr Val Glu
        130 135 140
Asp Ser Gly Thr Tyr Tyr Cys Thr Gly Lys Val Trp Gln Leu Asp Tyr
145 150 155 160
Glu Ser Glu Pro Leu Asn Ile Thr Val Ile Lys Ala
                                165 170
SEQ ID NO: 6
Met Ala Pro Ala Met Glu Ser Pro Thr Leu Leu Cys Val Ala Leu Leu
  1 5 10 15
Phe Phe Ala Pro Asp Gly Val Leu Ala Val Pro Gln Lys Pro Lys Val
                        20 25 30
Ser Leu Asn Pro Pro Trp Asn Arg Ile Phe Lys Gly Glu Asn Val Thr
                35 40 45
Leu Thr Cys Asn Gly Asn Asn Phe Phe Glu Val Ser Ser Thr Lys Trp
        50 55 60
Phe His Asn Gly Ser Leu Ser Glu Glu Thr Asn Ser Ser Leu Asn Ile
65 70 75 80
Val Asn Ala Lys Phe Glu Asp Ser Gly Glu Tyr Lys Cys Gln His Gln
85 90 95
Gln Val Asn Glu Ser Glu Pro Val Tyr Leu Glu Val Phe Ser Asp Trp
                                100 105 110
Leu Leu Leu Gln Ala Ser Ala Glu Val Val Met Glu Gly Gln Pro Leu
                        115 120 125
Phe Leu Arg Cys His Gly Trp Arg Asn Trp Asp Val Tyr Lys Val Ile
                130 135 140
Tyr Tyr Lys Asp Gly Glu Ala Leu Lys Tyr Trp Tyr Glu Asn His Asn
        145 150 155 160
Ile Ser Ile Thr Asn Ala Thr Val Glu Asp Ser Gly Thr Tyr Tyr Cys
                                165 170 175
Thr Gly Lys Val Trp Gln Leu Asp Tyr Glu Ser Glu Pro Leu Asn Ile
                        180 185 190
Thr Val Ile Lys Ala
                195
```

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Centigrade.

### Materials and Methods

Reagents. Human FcεRIα polypeptide was obtained from Heska Corp. and biotinylated as described below. Streptavidin coated ELISA plates were obtained from R&D systems. Phosphate Buffered Saline (PBS) and Bovine Serum Albumin (BSA) were azide free. Mouse anti-human IgE (ε-chain specific) antibody conjugated to alkaline phosphate (AP) was obtained from Sigma. Enzyme-linked immunosorbent assay (ELISA) wash (3 mM Tris, 0.1% Tween, 100 mM NaCl) was used in the washing steps of the assay.

Biotinylation. Thermo Fisher (Pierce) EZ-Link Sulfo-NHS-SS-Biotin kit was used to biotinylate FcεRIα following manufacturer's instructions.

ELISA Assay. Streptavidin coated ELISA plate was used (obtained from R and D systems). Biotinylated FcεRIα polypeptide was immobilized to the streptavidin coated plates via biotin by incubating the biotinylated FcεRIα polypeptide on streptavidin coated plates for half hour at room temperature (RT) with agitation (200 rpm). After the immobilization of FcεRIα polypeptide, a blocking solution (3 mM Tris, 0.1% Tween, 100 mM NaCl, 5% bovine serum albumin) was added and incubated for half hour. Following incubation with the blocking solution, the wells were washed in ELISA solution. The samples diluted in the blocking solution were added to the wells (100 μl/well) and incubated for one hour at RT at 200 rpm agitation. After further washing, anti-IgE AP conjugate (1:2000 dilution in blocking solution) were added to the wells and incubated for an hour at RT with agitation (200 rpm). After washing, p-NPP, substrate for AP was added (100 μl/well) and incubated for 25 minutes in dark. The reaction was stopped by addition of sodium hydroxide (1N NaOH). Absorbance was measured at 405 nm, corrected with values at 630 nm.

ImmunoCAP® Analyzer platform. The ImmunoCAP® Analyzer platform was used to conduct two types of assays for measuring IgE levels. ImmunoCAP® tubes contain a reaction chamber filled with a cellulose sponge matrix. A flexible hydrophilic polymer on which a protein of choice can be coated or covalently coupled.

The first assay was a conventional assay that measures total IgE using IgE assay kit (Phadia). The assay uses an anti-IgE monoclonal antibody coated onto the CAP matrix (the assay reaction tube). The serum samples were then added by the ImmunoCAP analyzer. After automatic washes, an anti-IgE conjugate was then added by the analyzer. A fluorescent signal was then generated for the sample, which was then compared to the values by the calibrators to calculate the concentration of total IgE for the sample.

The second assay was performed using CAP tubes coated with streptavidin (Phadia). Biotinylated FcεRIα polypeptide was added to streptavidin coated CAP tubes and incubated for half hour at RT. The subsequent steps were performed as per Phadia's ImmunoCAP® Analyzer protocol. Briefly, following washing to remove any unbound biotinylated FcεRIα polypeptide, serum samples were added and incubated for one hour at RT. After further washing, anti-IgE linked to a fluorophore was added (1:2000 dilution in blocking solution) were added to the wells and incubated for an hour at RT with agitation (200 rpm). A standard curve was used to derive the concentration of IgE antibody bound to biotinylated FcεRIα polypeptide.

### Example 1

### Use of Biotinylated Human FcεRIα Polypeptide Immobilized on a Substrate to Measure IgE

96-well ELISA plates were coated with three different amount of biotinylated FcεRIα polypeptide by applying 50 μL of biotinylated FcεRIα polypeptide at concentrations of 2, 5 and 20 μg/ml. Four IgE containing serum samples (samples 1-4, these samples diod not contain an anti-IgE therapeutic) were measured at no dilution (neat undiluted serum) and at 7-serial dilutions (½, ¼, ⅛, 1/16, 1/32, 1/64, and 1/128). Samples 1-4 had IgE had values of 497, 230, 101, and 53 IU/ml, respectively.

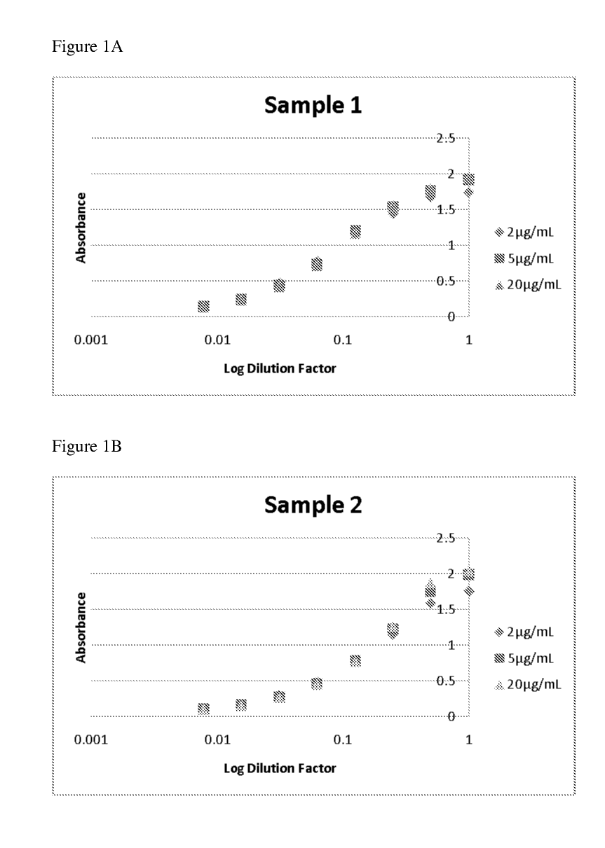

The results are shown in FIGS. 1A-D. Absorbance measured in wells coated with 2 μg/ml of biotinylated FcεRIα polypeptide (diamonds), 5 μg/ml of biotinylated FcεRIα polypeptide (squares), and 20 μg/ml of biotinylated FcεRIα polypeptide (triangles) illustrate the lack of matrix effect in this assay.

Matrix effect refers to a phenomenon in which free IgE cannot be accurately measured due to factors present in the sample matrix. Matrix effect can be detected by measuring a series of dilutions of a sample and plotting the concentration versus dilution factor. When the signal generated by the assay does not decrease as the sample is diluted, a matrix effect is detected.

Matrix effect is a common problem encountered in assays that detect an analyte in an undiluted or neat biological sample. As noted above, matrix effect is apparent when the analyte level measured in the undiluted sample does not correlate with the analyte levels measured in serial dilutions of the same sample. If the analyte level measured in the undiluted sample is correct, then as the sample is diluted a decrease in the analyte level in proportion to the fold-dilution of the sample should be observed. However, if the assay used to measure the analyte in the undiluted sample suffers from matrix effect, then the serial dilution of the sample will show an increase in the level of the analyte rather than a decrease. Accordingly, if an assay suffers from matrix effect then the analyte level cannot be reliably measured in an undiluted sample. On the other hand, if the serial dilution shows a corresponding decrease in analyte levels, then there is no matrix effect and the analyte level measured in the undiluted sample is correct.

The assay described herein does not suffer from matrix effect, as the response (absorbance) steadily decreased as the samples were serially diluted. Accordingly, this assay can be used to measure the levels of free IgE in an undiluted sample. There are numerous advantages to measuring free IgE levels in an undiluted sample. For example, measuring free IgE levels in an undiluted sample will enable measurement of very low levels of IgE antibodies, which level might be undetectable in the same sample once it has been even slightly diluted. Another advantage of an assay that can measure free IgE levels in an undiluted sample without matrix effects is that the assay provides a more accurate measurement of free IgE in the sample. This is because as the sample is diluted the complex formed by IgE and the anti-IgE therapeutic (for example, an anti-IgE antibody that binds to the CH3 domain of the IgE antibody) tends to dissociate resulting in an increase in the level of the free IgE antibodies. However, the level of free IgE measured in a diluted sample may not accurately reflect the levels of free IgE antibodies in the subject from whom the sample was obtained.

### Example 2

### Comparing Observed IgE Levels to Expected IgE Levels

A set of samples which did not contain anti-IgE therapeutics in the serum were chosen to assess the accuracy of IgE measurement of the assay using immobilized FcεRIα polypeptide. The total IgE concentration in these serum samples were measured by the Phadia's ImmunoCAP total IgE assay as described above and compared to the IgE concentration measured by this assay. Since the samples contained no anti-IgE therapeutics, the IgE concentrations measured by the two assays should be the same.

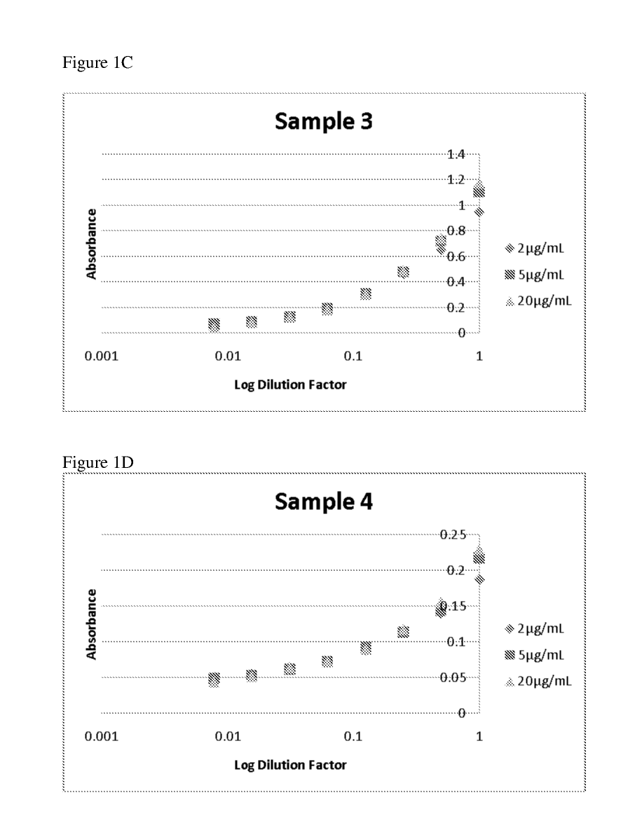

IgE levels in serum samples with known IgE concentrations (as determined by the Phadia's ImmunoCAP total IgE assay as described before) were measured using biotinylated FcεRIα polypeptide (0.214 per CAP tube). ImmunoCAP® tubes with the matrix coupled to streptavidin were obtained from Phadia. Biotinylated FcεRIα polypeptide was added to these tubes to immobilized biotinylated FcεRIα polypeptide in the tubes. Sample 1-3 were measured. FIGS. 2A-C illustrate the results. Linear regression line was drawn on the chart. R̂2 and the slope of the linear regression line reflects the extent of agreement between the total IgE and free IgE assays. Typically, slope of 0.8-1.2, and R̂2 of 0.8 or better represent good agreement between the two methods being compared. IgE levels were measured in 55 serum samples using the total IgE assay and compared to the IgE levels measured using immobilized biotinylated FcεRIα polypeptide. Since these serum samples do not contain therapeutic anti-IgE antibodies (for example omalizumab), both the assays were expected to provide similar measurements of IgE levels for all these samples.

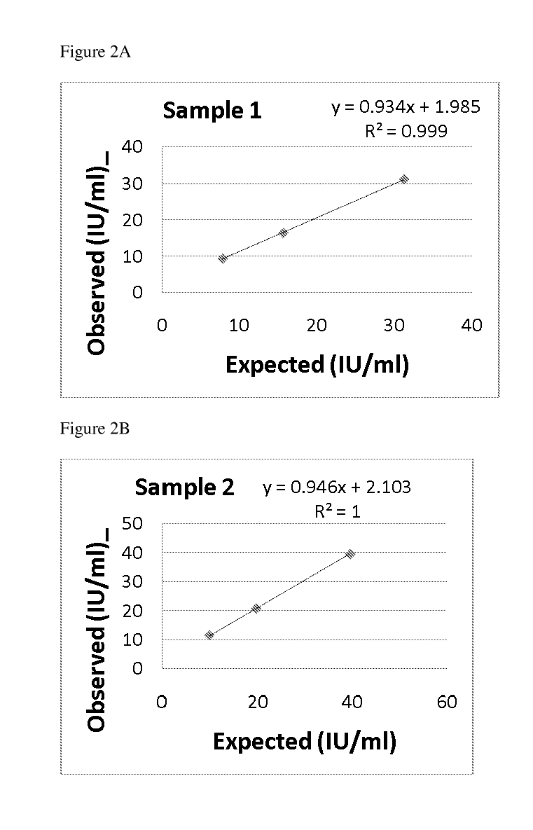

The results for the tested samples showed a high degree of agreement between the two methods (FIG. 3). The total IgE assay method is a well-established method (FDA approved), therefore, the agreement between the two methods indicates the accuracy of the immobilized FcεRIα polypeptide assay method for measuring IgE levels.

### Example 3

### Accuracy and Reproducibility of IgE Levels Measurement

Two samples were tested for determining the accuracy of the measurement of IgE level using immobilized FcεRIα polypeptide. Two samples were assayed in replicates of 3 or 5. Both samples were observed to show low CV % in concentration (see Table 1).

TABLE 1

| | |
|---|---|
| Sample 1 | Sample 2 |

| | |
|---|---|
| Sample | IgE (IU/ml) |

| | | |
|---|---|---|
| Replicate 1 | 7.3 | 126.6 |
| Replicate 2 | 7.1 | 121.6 |
| Replicate 3 | 6.7 | 116.1 |
| Replicate 4 | N.A. | 117.3 |
| Replicate 5 | N.A. | 121.8 |
| Mean | 7.0 | 120.7 |
| SD | 0.3 | 4.2 |
| CV % | 4.3 | 3.5 |

Five samples were tested for reproducibility of IgE measurements, using immobilized FcεRIα polypeptide, on different days. All five samples were observed to have relatively low CV % in concentration (see Table 2).

TABLE 2

| | Free IgE (IU/ml) | | | | |
|---|---|---|---|---|---|
| Day # | Sample 1 | Sample 2 | Sample 3 | Sample 4 | Sample 5 |
| Day 1 | 179.9 | 121 | 76.8 | 12.3 | 7.4 |
| Day 2 | 193.4 | 117.5 | 77.5 | 11.2 | 7.1 |
| Day 3 | 197.4 | 123 | 79.7 | 11.7 | 7.1 |
| Day 4 | 184.4 | 113.3 | 71.5 | 11.7 | 7 |
| Day 5 | 171.3 | 120.7 | 72.2 | 11.4 | 9.6 |
| Mean: | 185.4 | 119.1 | 75.5 | 11.7 | 7.6 |
| SD: | 10.5 | 3.8 | 3.5 | 0.4 | 1.1 |
| CV %: | 5.6 | 3.2 | 4.7 | 3.6 | 14.5 |

### Example 4

### Measurement of Free IgE

Three serum samples with total IgE between 100-500 IU/ml were incubated overnight with varying concentrations of Omalizumab (Xolair®) antibody (range of no Omalizumab to a 400-fold excess Omalizumab compared to IgE concentration). Total IgE and free IgE were measured in aliquots of the samples incubated with various amounts of the Omalizumab. Levels of total IgE antibody were measured using Phadia ImmunoCAP total IgE assay. Levels of free IgE were measured using immobilized FcεRIα polypeptide in the ImmunoCAP® Analyzer platform described above.

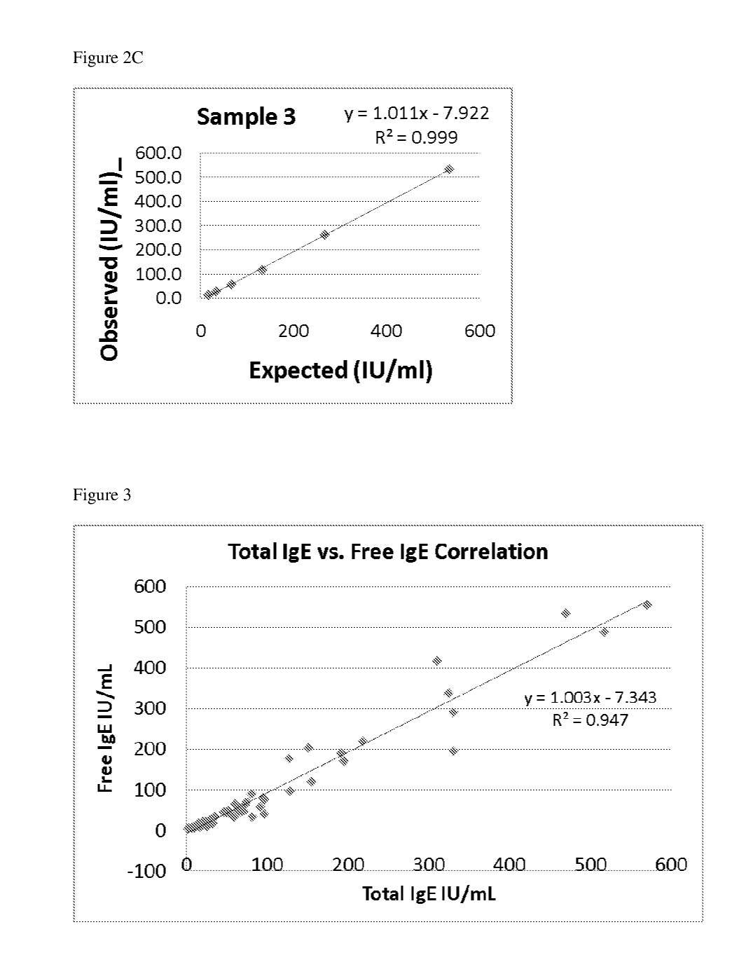

In brief, the total IgE assay uses an anti-IgE antibody which binds to all IgE molecules, free or those with its CH3 bound with an anti-IgE therapeutic, e.g., Omalizumab. After the capture of IgE molecules, another anti-IgE antibody which recognizes all IgE molecules, free or those with its CH3 bound with an anti-IgE therapeutic was used to detect IgE bound to the anti-IgE antibody immobilized on the substrate. The results shown in FIG. 4 illustrate that omalizumab up to 400 molar excess does not change the total IgE concentration.

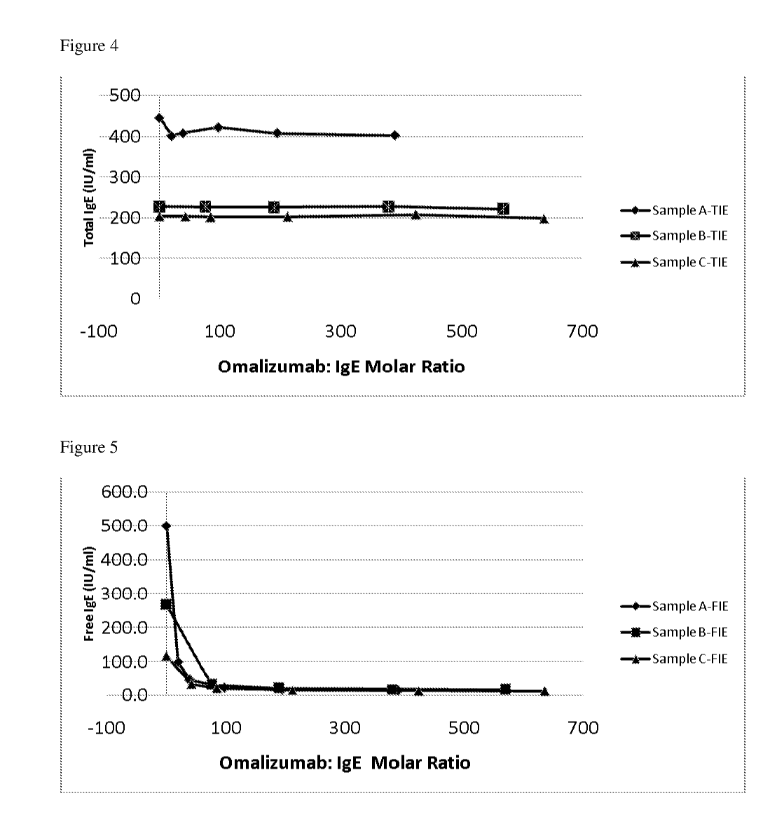

The free IgE assay only measures the fraction of IgE which is not bound by any anti-Ch3 domain IgE therapeutic such as omalizumab. As the samples were added with increasing concentration of omalizumab, the concentration of free IgE in the aliquots of the same sample are expected to decrease. The observed concentration did follow the expectation, showing a steady decrease in free IgE in a dose dependent manner for all three tested samples (FIG. 5). This decrease in free IgE levels with increasing concentrations of omalizumab illustrate that the assay is specifically measuring free IgE concentration.

What is claimed is:

1. A method for detecting free IgE antibody in a biological sample from a patient receiving an anti-IgE therapy, the method comprising:
a) contacting the biological sample with a capture reagent comprising at least an IgE Fc binding portion of FcεRIα polypeptide under conditions suitable for binding of free IgE antibody to the capture reagent thereby generating a free IgE antibody-capture reagent complex, wherein the capture reagent is immobilized onto a substrate via an anchoring compound that is directly or indirectly attached to the substrate;
b) contacting the capture reagent-free IgE antibody complex with a detection reagent that binds to a region of the free IgE not bound by the capture reagent; and
c) detecting the detection reagent bound to the capture reagent-free IgE antibody complex thereby detecting the presence or absence free IgE antibody in the sample.

2. The method of claim 1, wherein the anchoring molecule is indirectly attached to the substrate.

3. The method of claim 1, wherein the anchoring molecule is directly attached to the substrate.

4. The method of claim 2, wherein the anchoring molecule is a first member of a binding pair and the second member of the binding pair is attached to the substrate

5. The method of claim 4, wherein the first member is biotin and the second member is avidin or streptavidin.

6. The method of claim 3, wherein the anchoring molecule is an antibody that specifically binds to the capture reagent.

7. The method of claim 3, wherein the anchoring molecule is a bi-functional linker molecule covalently bound to the substrate and the capture reagent.

8. The method of claim 1, wherein the capture reagent comprises the anchoring compound and the anchoring compound is covalently attached to the capture reagent.

9. The method of claim 1, wherein the anti-IgE therapy comprises therapy with a polypeptide or peptide that binds to the same region of IgE as bound by the human FcεRIα polypeptide.

10. The method of claim 1, wherein the sample is an undiluted sample.

11. The method of claim 1, wherein the patient is a human.

12. A system for detecting free IgE antibody, the system comprising:
a capture reagent comprising at least an IgE Fc binding portion of FcεRIα polypeptide immobilized on a substrate via an anchoring compound that is directly or indirectly attached to the substrate; and
a biological sample from a subject receiving anti-IgE therapy.

13. The system of claim 12, comprising a detection reagent that binds to a region of the free IgE not bound by the capture reagent.

14. The system of claim 12, wherein the anchoring compound is indirectly attached to the substrate.

15. The system of claim 12, wherein the anchoring compound is directly attached to the substrate.

16. The system of claim 14, wherein the anchoring compound is a first member of a binding pair and the second member of the binding pair is attached to the substrate

17. The system of claim 16, wherein the first member is biotin and the second member is avidin or streptavidin.

18. The system of claim 15, wherein the anchoring molecule is an antibody that specifically binds to the capture reagent.

19. The system of claim 15, wherein the anchoring molecule is a bi-functional linker molecule covalently bound to the substrate and the capture reagent.

20. The system of claim 12, wherein the capture reagent comprises the anchoring compound.

21. The system of claim 12, wherein the anti-IgE therapy comprises therapy with a polypeptide or peptide that binds to the same region of IgE as bound by the human FcεRIα polypeptide.

22. The system of claim 12, wherein the biological sample is an undiluted sample.

23. The method of claim 12, wherein the subject is a human.
